(19) United States
(12) Patent Application Publication
(10) Pub. No.: US 2023/0233777 A1
(43) Pub. Date: Jul. 27, 2023

(54) CARBON DIOXIDE BASED METERED DOSE INHALER

(71) Applicant: KINDEVA DRUG DELIVERY L.P., Woodbury, MN (US)

(72) Inventors: John P. Bunting, Castle Donington (GB); Lee M. Hodges, Clitheroe (GB); Peter D. Hodson, Berkshire (GB); Stephen J. Howgill, Leicestershire (GB); Adam J. Stuart, Loughborough (GB); Kerry Ward, Rochdale (GB)

(73) Assignee: KINDEVA DRUG DELIVERY L.P., Woodbury, MN (US)

(21) Appl. No.: 17/779,028

(22) PCT Filed: Nov. 18, 2020
(86) PCT No.: PCT/US2020/061056

§ 371 Date: May 23, 2022

Related U.S. Application Data
(60) Provisional application No. 62/939,093, filed on Nov. 22, 2019.

(51) Int. Cl. A61M 15/00; A61M 16/20; A61M 16/00

(52) U.S. Cl. CPC A61M 15/0065; A61M 16/201; A61M 16/0003; A61M 2202/0225

(57) ABSTRACT

Various embodiments of a metered dose inhaler that includes a metering valve are disclosed. The inhaler includes a reservoir containing a pressurized formulation of medicament and carbon dioxide. The metering valve includes a metering chamber, a metering valve stem, and a metering valve stem seal having an opening through which the metering valve stem passes to form a dynamic seal between the metering valve stem and outside atmosphere. The metering valve stem seal has a Shore D hardness of 45 to 80 and its opening is adapted to be stretched wider by the metering valve stem passing through it than it would be absent the valve stem.

This application claims the benefit of U.S. Provisional Application No. 62/939,093, filed Nov. 22, 2019, and U.S. Provisional Application No. 62/962,018, filed Jan. 16, 2020, the disclosures of which are incorporated by reference herein in their entireties.

### FIELD

The present disclosure generally relates to medicinal inhalers and, more particularly, to medicinal inhalers that include carbon dioxide as a propellant.

### BACKGROUND

Delivery of aerosolized medicament to the respiratory tract for the treatment of respiratory and other diseases can be done using, by way of example, pressurized metered dose inhalers (pMDI), dry powder inhalers (DPI), or nebulizers. pMDIs are familiar to many patients who suffer from asthma or from chronic obstructive pulmonary disease (COPD). pMDI devices can include an aluminum canister, sealed with a metering valve, that contains medicament formulation. Generally, the medicament formulation is a solution and/or suspension of one or more medicinal compounds in a liquefied hydrofluoroalkane (HFA) propellant.

In pulmonary pMDIs, the sealed canister can be provided to the patient in an actuator—a generally L-shaped plastic part that includes a generally vertical tube that surrounds the canister plus a generally horizontal tube that forms a patient portion (e.g., a mouthpiece or nosepiece) that can define an inspiration (or inhalation) orifice.

The canister typically includes a metering valve that is crimped onto an appropriately-sized metal can. The metal can is typically made of aluminium, having a wall thickness of approximately 0.5 mm. The canister contains a formulation typically including liquid propellant(s), drug(s), co-solvent(s) and excipient(s). To prevent loss of the formulation, (primarily the liquid propellant), the metering valve contains rubber components that form seals.

Historically, the propellants in most pMDIs had been chlorofluorocarbons (CFCs). However, environmental concerns during the 1990s led to the replacement of CFCs with hydrofluoroalkanes (HFAs) as the most commonly used propellant in pMDIs. Although HFAs do not cause ozone depletion they do have a stated high global warming potential (GWP), which is a measurement of the future radiative effect of an emission of a substance relative to that of the same amount of carbon dioxide (CO2). The two HFA propellants most commonly used in pMDIs are HFA134a (CF3CH2F) and HFA 227 (CF3CHFCHF3) having stated 100-year GWP values of 1300 to 1430 and 3220 to 3350, respectively.

Various other propellants have been proposed over the years. Among them, carbon dioxide (CO2) has been mentioned as a potential propellant for pMDIs, but no pMDI product has been successfully developed and commercialized using carbon dioxide as a propellant.

### SUMMARY

It has now been found that despite CO2's major differences from other MDI propellants (such as much higher vapor pressure and different density, polarity, solubility, and component interaction characteristics) a practical pMDI can be made using CO2. This can be very useful due to CO2's stated lower GWP (GWP value of 1).

In particular, it has been found that conventional pMDI metering valves can have serious problems with CO2-containing pMDIs, such as, by way of example, leakage and valve performance. The valve stem seal, which is a dynamic seal through which the valve stem slides during actuation, can be particularly problematic. However, it has also now been found that using significantly harder stem seal materials, having a Shore D hardness of 45 to 80, and a valve stem seal opening sized sufficiently smaller than the valve stem passing through it so that the valve stem stretches the hard seal material, can address these problems, providing significant performance and leak prevention benefits. Moreover, contrary to the teachings of U.S. Pat. No. 6,032,836, this can be achieved without the need to use a lip seal design with an integral spring.

It has further been found that, unlike conventional pMDI valves, metered dose valves of the various embodiments disclosed herein can be made with an axial (relative to the axis of the MDI canister and motion of actuation) valve stem movement, but without using a spring to return the valve stem after actuation, instead relying on the increased pressure of the CO2 to do so.

Accordingly, some embodiments of the present disclosure provide a pMDI including a reservoir containing a pressurized formulation of medicament and CO2, and equipped with a metering valve having a metering chamber, a metering valve stem, and a metering valve stem seal having an opening through which the metering valve stem passes to form a dynamic seal between the metering valve stem and outside atmosphere. The metering valve stem seal has a Shore D hardness of 45 to 80 and its opening is adapted to be stretched wider by the metering valve stem passing through it than it would be absent the valve stem.

Some embodiments provide a pMDI having a reservoir containing a pressurized formulation of medicament and CO2. The pMDI is equipped with a valve including a valve housing having a first end, a second end exposed to pressurized formulation in the reservoir, and walls defining a metering chamber for receiving the formulation from the reservoir. The valve further includes a metering valve stem located within the metering chamber, the metering valve stem having a first end exposed to atmosphere and a second end exposed to pressurized formulation in the reservoir. At least a portion of the metering valve stem proximate the first end has a diameter larger than the diameter of at least a portion of the metering valve stem proximate the second end. The valve further includes a metering valve stem seal member in contact with the first end of the valve housing and the metering valve stem, where the metering valve stem seal member has a Shore D hardness of about 45 to 80. The valve has a primed position where the formulation can freely flow between the reservoir and the metering chamber and where the metering valve stem seal member seals the metering chamber from the outside atmosphere. The valve has an actuated position where the second end of the metering valve stem seals against the second end of the valve housing to seal the metering chamber from the reservoir, thereby defining a metered volume of formulation within the metering chamber, and where the metering valve stem includes a flow path allowing the formulation to freely flow between the metering chamber and the outside atmosphere.

Some embodiments of the present disclosure provide a pMDI having a reservoir containing a pressurized formulation of medicament and CO2. The pMDI is equipped with a valve including a valve housing having a first end, a second end with an opening exposed to pressurized formulation in the reservoir, and walls defining a metering chamber for receiving the formulation from the reservoir. The valve further includes a metering valve stem located within the metering chamber, the metering valve stem having a first end exposed to atmosphere and a second end exposed to pressurized formulation in the reservoir. At least a portion of the metering valve stem proximate the first end has a diameter larger than the diameter of at least a portion of the metering valve stem proximate the second end. The valve further includes a metering valve stem seal member in contact with the first end of the valve housing and the metering valve stem, where the metering valve stem seal member has a Shore D hardness of about 45 to 80 and does not include a spring. The valve has a primed position where the formulation can freely flow between the reservoir and the metering chamber, and where the metering valve stem seal member seals the metering chamber from the outside atmosphere. The valve has an actuated position where the opening at the second end of the valve housing is sealed from the reservoir, thereby defining a metered volume of formulation within the metering chamber, and where the metering valve stem includes a flow path allowing the formulation to freely flow between the metering chamber and the outside atmosphere.

The present disclosure also provides inhalers including valves of the embodiments described above where the inhaler further includes a canister having a reservoir and an actuator including an actuator housing configured to enclose at least a portion of the valve and canister.

Some embodiments of the present disclosure provide an inhaler including a canister having a reservoir containing a pressurized formulation of medicament and CO2. The inhaler further includes a metering valve having a valve housing having a first end, a second end exposed to pressurized formulation in the reservoir, and walls defining a metering chamber for receiving the formulation from the reservoir. The metering valve further includes a metering valve stem located within the metering chamber, the metering valve stem having a first end exposed to atmosphere and a second end exposed to pressurized formulation in the reservoir. The metering valve further includes a metering valve stem seal member in contact with the first end of the valve housing, where the metering stem seal has a Shore D hardness of about 45 to 80. The valve has a primed position where the formulation can freely flow between the reservoir and the metering chamber and where the metering valve stem seal member seals the metering chamber from the outside atmosphere. The valve has an actuated position where the opening at the second end of the valve housing is sealed from the reservoir, thereby defining a metered volume of formulation within the metering chamber, and where the metering valve stem includes a flow path allowing the formulation to freely flow between the metering chamber and the outside atmosphere. The canister has a lower canister body, a portion of the lower canister body forming the first end of the valve housing and having an opening configured to accept the metering valve stem. The metering valve stem has a retention feature that is sized so that it cannot pass through the lower canister body opening. The inhaler further includes an actuator including an actuator housing configured to enclose at least a portion of the valve and canister.

Other features and aspects of the present disclosure will become apparent by consideration of the detailed description and accompanying drawings.

### BRIEF DESCRIPTION OF THE DRAWINGS

FIG. 1 is a side view of an inhaler including a canister containing a valve according to embodiments of the present disclosure.

FIG. 2 is a side view of a cross-section of the inhaler of FIG. 1 taken along the centerline of the inhaler.

FIG. 3 is an exploded view of the canister of FIG. 1.

FIG. 4 is an enlarged side view of a cross-section of the canister of FIG. 1.

FIG. 5 is an enlarged isometric view of a cross-section of the lower portion of the canister with a valve depicted in its primed position.

FIG. 6 is an enlarged isometric view of a cross-section of the lower portion of the canister with a valve depicted in its actuated position.

FIG. 7 is an enlarged view of the valve in its primed position.

FIG. 8a is a cross-sectional view of a sealing member according to embodiments of the present disclosure.

FIG. 8b is a cross-sectional view of a sealing member according to embodiments of the present disclosure.

FIG. 8c is a cross-sectional view of a sealing member according to embodiments of the present disclosure.

FIG. 8d is a cross-sectional view of a sealing member according to embodiments of the present disclosure.

FIG. 9 is a cross-sectional view of a metering valve according to embodiments of the present disclosure.

FIG. 10 is a cross-sectional view of a metering valve according to embodiments of the present disclosure.

### DETAILED DESCRIPTION

Throughout this disclosure, singular forms such as “a,” an,” and “the:” are often used for convenience; singular forms are meant to include the plural unless the singular alone is explicitly specified or is clearly indicated by the context. Numerical ranges, for example “between x and y” or “from x to y”, include the endpoint values of x and y.

Some terms used in this application have special meanings, as defined herein. All other terms will be known to the skilled artisan and are to be afforded the meaning that a person of skill in the art at the time of the invention would have given them.

Elements in this specification that are referred to as “common,” “commonly used,” “conventional,” “typical,” “typically,” and the like, should be understood to be common within the context of the compositions, articles, such as inhalers and pMDIs, and methods of this disclosure; this terminology is not used to mean that these features are present, much less common, in the prior art. Unless otherwise specified, only the Background section of this Application refers to the prior art.

The present disclosure will be described with respect to particular embodiments and with reference to certain drawings, but the invention is not limited thereto. The drawings described are only schematic and are non-limiting. In the drawings, the size of some of the elements can be exaggerated and not drawn to scale for illustrative purposes.

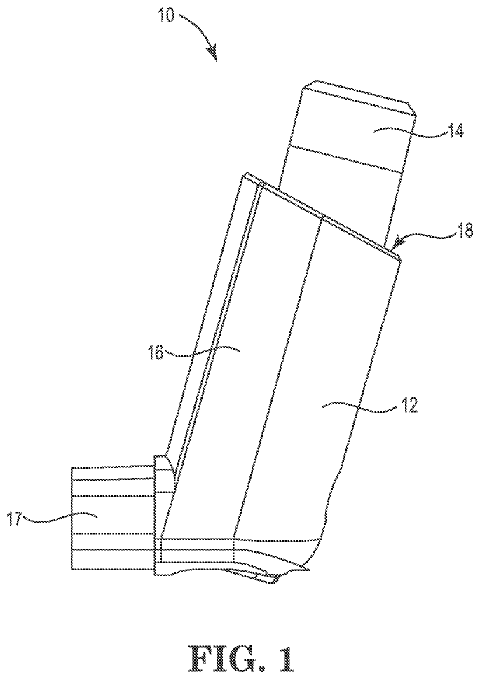

FIG. 1 depicts a pMDI 10 including an actuator 12 and a canister 14. Actuator 12 has a generally elongate actuator body 16 that acts as a housing for canister 14. Canister 14 is inserted into canister opening 18 at the top of actuator 12. Canister 14 is pressurized and contains a medicament formulation for delivery to a user via actuator 12 and mouthpiece 17 as will be described in further detail herein. In other embodiments mouthpiece 17 can be replaced by a nosepiece (not depicted) to enable nasal delivery.

The medicament formulation includes one or more active pharmaceutical ingredients and liquid CO2. The CO2 acts a propellant to propel the formulation from canister 14 into mouthpiece 17 and then to the patient. The formulation can further include one or more excipients. In some embodiments one or more of the active pharmaceutical ingredients and excipients can be dissolved in the liquid CO2. In some embodiments one or more of the active pharmaceutical ingredients and excipients can be dispersed or suspended in the liquid CO2.

The medicament formulation can include any suitable pharmaceutical ingredients or medicinal compositions, e.g., one or more of the medicinal compositions described in U.S. Provisional Application No. 62/962,018, filed Jan. 16, 2020, and entitled MEDICINAL COMPOSITIONS FOR CARBON DIOXIDE BASED METERED DOSE INHALERS.

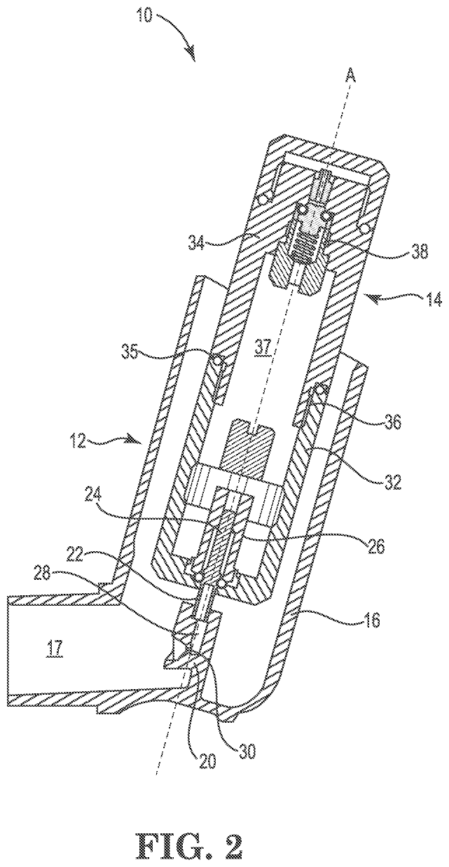

Turning now to FIG. 2, actuator 12 includes body 16 having stem post 20. The stem post 20 has stem socket 22 for receiving metering valve stem 24 of metering valve 26. The stem post 20 also defines expansion chamber 28, which delivers medicament formulation into mouthpiece 17 via jet orifice 30.

The canister 14 has a generally cylindrical form with upper housing 34 and lower housing 32 connected by screw thread 36 and sealed by canister seal member 35 to form reservoir 37 for holding the medicament formulation. In some embodiments the screw thread attachment can be replaced by another attachment, such as welding and/or gluing together of the upper housing 34 and lower housing 32. The upper housing 34 includes pressure fill valve 38 to enable filling of the canister 14 with medicament formulation. The lower housing 32 of canister 14 houses a portion of the metering valve 26. The reservoir 37 and metering valve stem 24 share a central longitudinal axis A.

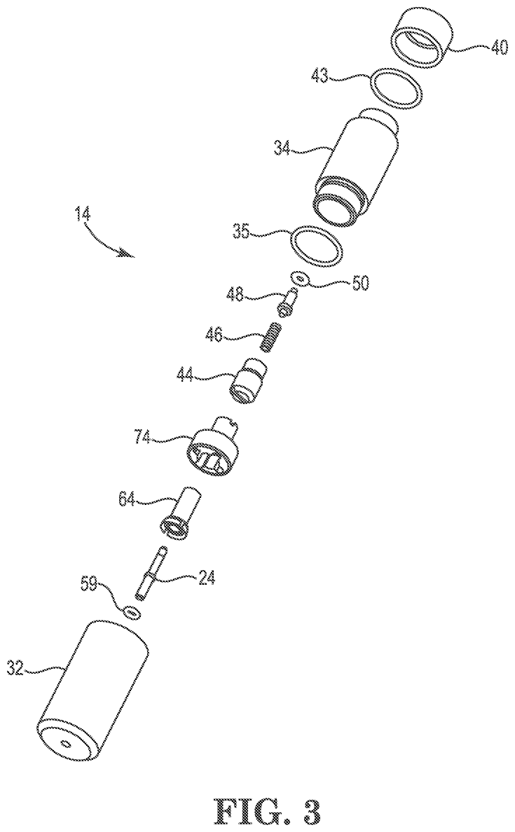

In FIG. 3 the canister 14 is depicted in further detail to include lower housing 32, metering valve stem 24, metering valve stem seal member 59, valve housing 64, valve clamp 74, retaining bolt 44, spring 46, pressure fill valve stem 48, pressure fill valve stem seal member 50, canister seal member 35, the upper housing 34, fill valve seal member 43, and fill valve cap 40.

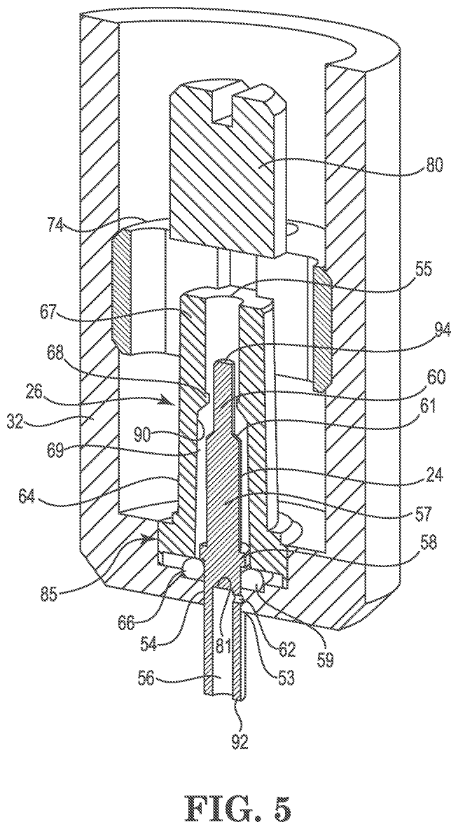

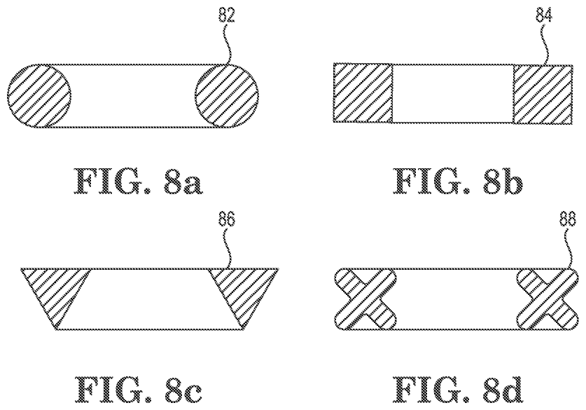

In any embodiment, one or more of the sealing members, including canister seal member 35, metering valve stem seal member 59, pressure fill valve stem seal member 50, canister seal member 35, fill valve seal member 43, and a narrowed internal portion 68 (FIG. 5) of the valve housing 64 can be in the form of a ring or a torus or a disk having a central, circular opening. Exemplary shapes are illustrated in FIGS. 8a-d and include an O-ring having a circular cross-section 82, a square cut ring or planar annular seal having a rectangular cross-section 84, a delta- or triangular shaped- ring having a triangular cross-section 86, and an X- or quad-ring having an X-shaped cross-section 88.

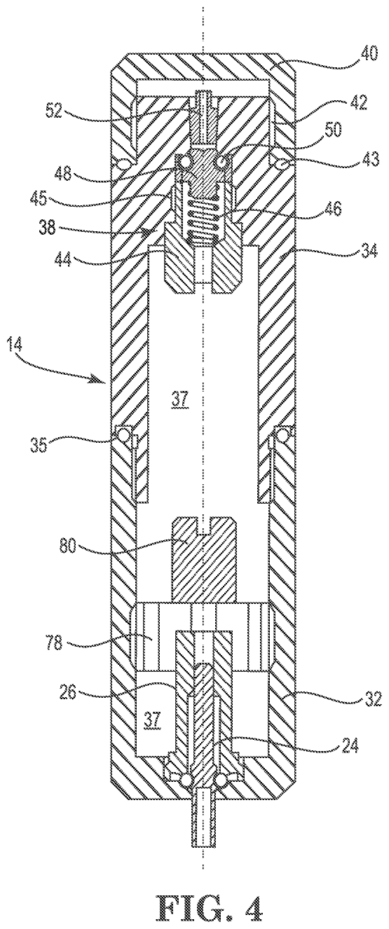

In FIG. 4, the fill valve cap 40 is depicted connected to the uppermost end of the upper housing 34 by screw thread 42 and is sealed by fill valve seal member 43. In some embodiments the screw thread attachment can be replaced by another attachment, such as welding or gluing together of the fill valve cap 40 and upper housing 34. The cap 40 covers fill port 52 to provide the user with a surface to press to actuate the inhaler 10 as will be described further herein. The pressure fill valve 38 includes retaining bolt 44, which threads into internal port 45 of upper housing 34. The retaining bolt 44 acts against a lower end of spring 46 to urge the pressure fill valve stem 48 against the pressure fill valve stem seal member 50 to seal the reservoir 37. An upper end of the pressure fill valve stem 48 defines fill port 52 for receiving the pressurized medicament formulation.

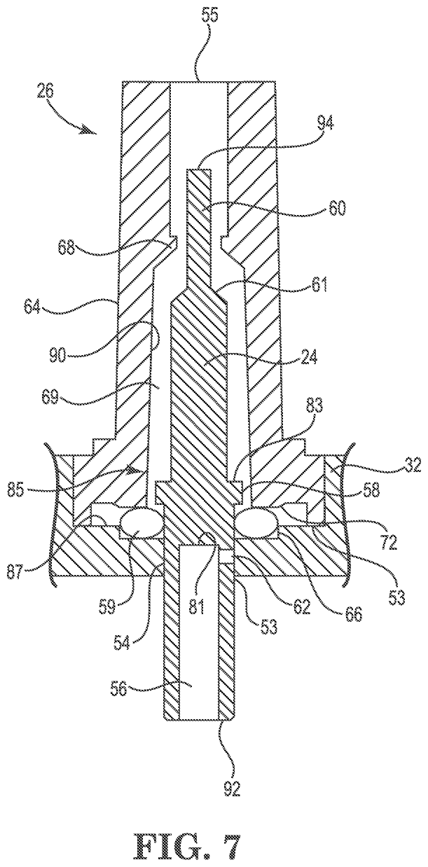

FIGS. 5 to 7 depict detailed views of the metering valve 26. The lower housing 32 of the canister 14 has a port 54 through which the metering valve stem 24 of the metering valve 26 extends. The metering valve stem 24 has a generally cylindrical shape with a blind hole 56 at its lower or first end 92, an outwardly extending circumferential abutment 58 at a mid-section 57, and a pin section 60 of reduced diameter at its upper or second end 94. The blind hole 56 provides an outlet for the medicament formulation into the actuator 12. The metering valve stem 24 has a radial port 62 extending from the blind hole 56 though the wall of the metering valve stem 24 at a position proximate a base 81 of the blind hole 56. The abutment 58 engages with an annular seal in the form of metering valve stem seal member 59 to form a seal between the metering valve stem 24, the valve housing 64, and the canister lower housing 32 when the inhaler 10 is in the primed condition as depicted in FIGS. 4 and 5. In one or more embodiments, a surface area of a surface 83 (FIG. 7) of the abutment 58 that faces a metering chamber 69 of the valve 26 can be selected to increase the effectiveness of the seal between the abutment and the metering valve stem seal member 59. Such selection can be based on a balancing between sufficient force to create an effective seal between surface 83 and abutment 58 and an actuation force required to actuate the valve 26 such that a user does not struggle to actuate the valve. For example, Table 1 illustrates Force to Fire for several values of surface area for the metering-chamber-facing surface 83 of the abutment 58 for a given formulation pressure.

TABLE 1

| | Surface | Force |
|---|---|---|
| Formulation | Area | to Fire |
| Pressure (bar) | (mm²) | (N) |
| 50 | 12 | 60 |
| | 11 | 55 |
| | 10 | 50 |
| | 9 | 45 |
| | 8 | 40 |
| | 7 | 35 |
| 55 | 12 | 66 |
| | 11 | 60.5 |
| | 10 | 55 |
| | 9 | 49.5 |
| | 8 | 44 |
| | 7 | 38.5 |
| 60 | 12 | 72 |
| | 11 | 66 |
| | 10 | 60 |
| | 9 | 54 |
| | 8 | 48 |
| | 7 | 42 |

As can be seen in Table 1, a surface area of 8 mm² for the surface 83 of the abutment 58 for an inhaler having a formulation pressure of 50 bar would provide a Force to Fire of about 40N.

The mid-section 57 and pin section 60 of the metering valve stem 24 are situated within the valve housing 64, which has a metering chamber 69 open at both a first end 53 and a second end 55 of the valve housing. The valve housing 64 has an upper section 67 proximate the second end 55 with internal dimensions that allow for clearance between the pin section 60 of the metering valve stem 24 and the valve housing 64 when the valve 26 is in the primed position, while forming an interference fit between the mid-section 57 and the valve housing 64 when the valve is in the actuated position. In particular, the valve stem 24 transitions from the mid-section 57 to the pin section 60 via a beveled surface 61, and the valve housing upper section 67 includes narrowed internal portion 68 that is dimensioned to form an interference fit with the mid-section 57 of the valve stem. The narrowed internal portion 68 acts as a seal member against the mid-section 57 of the valve stem 24. The metering chamber 69 is disposed between the metering valve stem 24, the valve housing 64, and the metering valve stem seal 59 when in the actuated position (or more particularly, just prior to reaching the actuated position as the valve transitions from primed to actuated position). The size of the metering chamber 69 defines a metered volume that can be about 25 to 100 microliters, 25 to 80 microliters, 40 to 65 microliters, 25 microliters, 50 microliters, or 62 microliters. A lower section 85 of the valve housing 64 has a circumferential step 66, which is formed in an inner face 87 (FIG. 7) of the lower housing 32. Opposed to the step 66 is a protrusion 72 (FIG. 7). In some embodiments, the step 66 and protrusion 72 act to provide positional stability to the metering valve stem seal member 59. In some embodiments, the step 66 and/or protrusion 72 can be omitted.

An upper or second end 55 of valve housing 64 is urged towards the lower end of the canister 14 by a valve retainer in the form of the valve clamp 74, which has an external screw thread (not shown), and which engages an internal screw thread (also not shown) on the inner surface of the lower housing 32. Any suitable technique or techniques can be utilized to connect the valve clamp 74 and the lower housing 32, e.g., threads, interference fit, adhesives, etc. The valve clamp 74 has a central opening, which permits flow of the medicament formulation within the reservoir 37 and from the reservoir into the upper or second end 55 of the metering chamber 69 of the valve. The valve clamp 74 has an integral bolt 80 to facilitate the tightening of the upper section 67 of the valve housing 64 against the lower housing 32. Alternative techniques of attaching the upper section 67 of the valve housing 64 against the lower end of the canister 14 can be employed for example, an interference fit, welding, and/or gluing.

Alternative flow paths to allow for medicament formulation to flow from the reservoir 37 into the second end 55 of the metering chamber 69 of the valve 26 can be used. For example, a bottle emptier could surround the exterior of the valve housing 64 so that formulation would flow from the base of the canister 14, up the side of the valve housing, and then down into the second end 55 of the valve housing and the metering chamber 69. Alternatively, one or more openings into the metering chamber 69 could be in the sidewall of the upper section 67 of the valve housing 64 at a point above the narrowed internal portion 68.

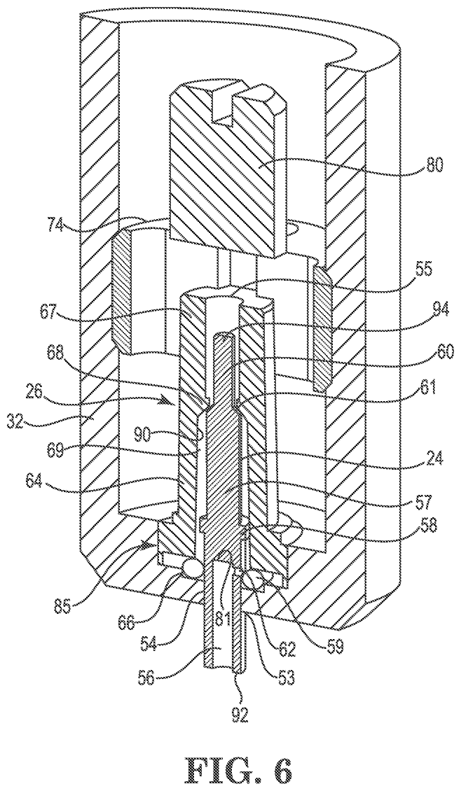

The opening of the metering valve 26 will now be considered in further detail with reference to FIGS. 5 and 6. In FIG. 5, the valve 26 is in its primed position with the valve stem radial port 62 positioned below the metering valve stem seal member 59. There is clearance between the pin section 60 of the metering valve stem 24 and the second end 55 of the valve housing 64 and into the metering chamber 69. As a result, the metering chamber 69 is filled with medicament formulation.

In FIG. 6, the inhaler 10 has been actuated by the patient, causing the canister 14 (not depicted) to move downwardly relative to the metering valve stem 24, which has remained in position by virtue of its engagement with the stem socket 22 (also not depicted). As a result of the movement of the canister 14, the valve stem radial port 62 is positioned above the metering valve stem seal member 59, allowing flow of the medicament formulation from the metering chamber 69 through the valve stem radial port 62 and into the actuator via the blind hole 56 at the lower end of the metering valve stem 24. The mid-section 57 of the metering valve stem 24 is now abutting walls 90 of the valve housing 64 at the narrowed internal portion 68 of the upper section 67. In a preferred embodiment, the walls 90 of the valve housing 64 at the upper section 67 are flexible to aid in forming a seal as the mid-section 57 and the beveled surface 61 of the valve stem 24 interacts with them. As a result, no flow of medicament formulation from the reservoir 37 is permitted into the metering chamber 69 until the patient releases the canister 14. Upon release of the manual force upon the canister 14, the pressure within the reservoir 37 causes the canister and valve 26 to return to their primed positions (depicted in FIG. 5 for the valve), thereby allowing the metering chamber 69 to once again fill with medicament formulation such that it is ready for subsequent use.

Exemplary materials used to form the sealing members, in particular, the metering valve stem seal member 59, include, but are not limited to, polytetrafluoroethylene (PTFE), ethylene tetrafluoroethylene (ETFE), fluorinated ethylene propylene (FEP), polyethylene, particularly high-density polyethylene or linear low-density polyethylene, polyamide, such as nylon, and polypropylene. The metering valve stem seal member 59 can have a hardness of about 45 to 80, 50 to 70, 50 to 60, or 55 to 60 Shore D as measured by ASTM D2240-15 in a standard atmosphere of 23° C. and 50% relative humidity. In embodiments all of the seal members, including for example the canister seal member 35, metering valve stem seal member 59, pressure fill valve stem seal member 50, canister seal member 35, and fill valve seal member 43 are all formed from the same material. In embodiments the metering valve stem seal member 59 is formed from PTFE. In embodiments the narrowed internal portion 68 of the valve housing 64 is high-density polyethylene.

The valve stem 24 preferably has a hardness equal to or greater than the hardness of the metering valve stem seal member 59 and/or the narrowed internal portion 68 of the valve housing. Suitable valve stem hardness values are about greater than 45, greater than 50, greater than 60, greater than 70, greater than 80, 50 to 120, 60 to 110, or 70 to 100 Shore D as measured by ASTM D2240-15 in a standard atmosphere of 23° C. and 50% relative humidity. In some embodiments the hardness of the valve stem 24 is only equal to or greater than the hardness of the metering valve stem seal member 59 and/or the narrowed internal portion 68 of the valve housing 64 in the region of the valve stem that contacts each respective portion. Exemplary materials used to form the valve stem 24 include, but are not limited to, nylon, polyacetal or polyoxymethylene, and stainless steel.

In embodiments the metering valve stem seal member 59 has an internal diameter that is smaller than the external diameter of the portion of the valve stem 24 that interacts with the metering valve stem seal member 59. The internal diameter of the metering valve stem seal member 59 can be about 2.0 to 4.0, 2.0 to 3.0, 2.5 to 2.7, or 2.57 mm. The cross section can be about 1.0 to 3.0, 1.5 to 2.0, 1.7 to 1.9, or 1.78 mm. The cross-section is the width of the metering valve stem seal member 59 in the direction perpendicular to the valve stem 24, for example, the diameter of an O-ring. The external diameter of the portion of the valve stem 24 that interacts with the metering valve stem seal member 59 can be about 2.2 to 4.4, 2.2 to 3.3, 2.7 to 3.0, or 2.8 mm. In some embodiments the difference between the external diameter of the valve stem portion that interacts with the metering valve stem seal member 59 and the internal diameter of the metering valve stem seal member 59 is about 0.2 to 0.4, 0.2 to 0.3, or 0.23 mm. In some embodiments the ratio of the external diameter of the valve stem portion that interacts with the metering valve stem seal member 59 and the internal diameter of the metering valve stem seal member can be about 1.02 to 1.20, 1.05 to 1.15, 1.08 to 1.12, or 1.10

In some embodiments, one or more of the sealing members, with the exception of the metering valve stem seal member 59, in the pMDI 10, including canister seal member 35, pressure fill valve stem seal member 50, canister seal member 35, and fill valve seal member 43, can be formed of a rubber material, such as nitrile rubber, ethylene propylene diene (EPDM) terpolymer, or butyl rubber.

The canister 14 is generally pressurized to between 45 and 80 bar, often to between 50 and 70 bar, and sometimes to between 50 and 60 bar. In general, the canister 14 is configured to withstand a pressure of at least 80 Bar. The pressure is selected so that at least a portion of the CO2 in the canister 14 is present as a liquid. The total amount of formulation is desirably selected so that at least a portion of the CO2 in the canister 14 is present as a liquid after a predetermined number of medicinal doses have been delivered. The predetermined number of doses can be 30 to 200, 60 to 200, 60 to 120, 60, 120, 200, or any other suitable number of doses. The total amount or volume of liquid formulation in the canister 14 can be about 1 to 30 mL, 2 to 25 mL, 5 to 20 mL, or 10 to 20 mL. The total amount of formulation is typically selected to be greater than the product of the predetermined number of doses times the metering volume of the metered valve. This helps to ensure that the amount of each dose remains relatively constant through the life of the inhaler.

The process of filling the canister 14 will now be considered. The liquid medicament formulation is pumped through the pressure fill valve 38 at a pressure of 45 to 80 bar, 45 to 70 bar, 45 to 60 bar, 45 bar, 50 bar, 55 bar, or 60 bar. It flows through the pressure fill valve 38 and into the reservoir 37. It then passes into the second end 55 of the valve housing 64 and into the metering chamber 69. By virtue of the clearance between the pin section 60 of the metering valve stem 24 and the inner wall 90 of the metering chamber 69 of the valve housing 64 the medicament formulation is permitted to pass into the metering chamber. The medicament formulation is prevented from leaving the canister 14 by the seal formed by the metering valve stem seal member 59 when the valve is in its primed, or rest, position.

In use, the patient actuates the inhaler 10 by pressing downwardly on the end cap 40 of the canister 14. This moves the canister 14 into the body 16 of the actuator 12 and presses the canister metering valve stem 24 against the stem socket 22, resulting in the canister metering valve 24 opening and releasing a metered dose of medicament into the expansion chamber 28 within the stem post 20. The expansion chamber 28 delivers the dose into the mouthpiece 17 via the jet orifice 30 and from there the dose passes into the patient's mouth. It should be understood that other modes of actuation, such as breath-actuation, can be used as well and would operate as described with the exception that the force to depress the canister would be provided by the device, for instance by a spring or a motor-driven screw, in response to a triggering event, such as patient inhalation.

In embodiments, the canister 14 is urged from the actuated position to the primed position by the pressurized propellant alone and without the aid of a return spring. In contrast, other pMDIs use a spring in the valve to bias the valve stem towards the primed position. Other inhalers are also known where springs are employed to assist with the sealing elements of the valve. In embodiments, the sealing elements of the valve 26 are not assisted by springs. Thus, the valve 26 can act as a springless valve (i.e., neither stem bias nor seal assist springs used). This can be advantageous as it reduces part count and eliminates components that can be susceptible to drug deposition. Furthermore, inclusion of a spring will increase the force to actuate the valve 26, as the user will need to overcome the force exerted on the valve stem 24 by both formulation and the spring.

It will be appreciated that in the above exemplary embodiment, the metering valve 26 is situated within the canister internal storage volume. It is entirely feasible within the scope of the disclosure that some or all of the metering valve 26 be positioned externally to the storage volume, for example by use of a manifold through which medicament formulation could be delivered to the metering valve.

The metering valve 26 can have any suitable leak rate. In one or more embodiments, the metering valve 26 can have a leak rate of no greater than 725 mg/yr. Further, in one or more embodiments, the metering valve 26 can have a leak rate of no greater than 525 mg/yr.

Some valves include a tank or reservoir seal disposed within a valve housing of the valve. In such valves, the reservoir seal can become displaced from its desired location adjacent to a second end of the valve housing, thereby diminishing the effectiveness of the seal in preventing pressurized formulation from entering the valve between an opening in the housing and a valve stem. This leakage of formulation can affect shot weights of the valve. To overcome this potential displacement of the seal, a spring can be disposed within the housing that engages the seal and holds the seal in place; however, such spring can make operation of the valve more challenging for users as it increases the Force to Fire of the inhaler.

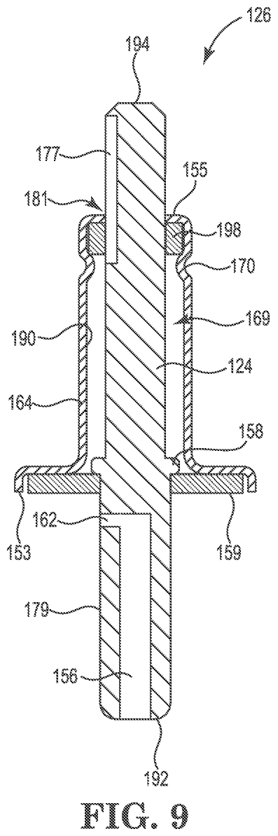

In one or more embodiments of valves described herein, one or more techniques can be utilized to prevent displacement of the reservoir seal during use without the need for a spring. For example, FIG. 9 is a cross-sectional view of another embodiment of a metering valve 126. All of the design considerations and possibilities described herein regarding metering valve 26 of FIGS. 1-8 apply equally to metering valve 126 of FIG. 9. The metering valve 126 can be utilized with any metered dose inhaler described herein, e.g., inhaler 10 of FIGS. 1-2.

The metering valve 126 includes a valve housing 164 that has a first end 153, a second end 155 exposed to pressurized formulation in a reservoir (e.g., reservoir 37 of FIG. 2) of an inhaler (e.g., inhaler 10 of FIG. 2), and walls 190 that define a metering chamber 169 for receiving the formulation from the reservoir.

The metering valve 126 also includes metering valve stem 124 located within the metering chamber 169. The metering valve stem 124 includes a first end 192 exposed to atmosphere and a second end 194 exposed to pressurized formulation in the reservoir. A metering valve stem seal member 159 is in contact with the first end 153 of the valve housing 164 and the metering valve stem 124. A fill channel 177 is disposed in the metering valve stem 124 adjacent to the second end 194 of the stem. The fill channel 177 can be adapted to allow pressurized formulation from the reservoir to pass through the fill channel and into the metering chamber 169 when the fill channel extends between the reservoir and the metering chamber. The fill channel 177 can take any suitable shape or shapes and have any suitable dimensions. The metering valve stem 124 also includes a radial port 162 that extends from an outer surface 179 of the valve stem 124 to a blind hole 156 disposed in the valve stem. The blind hole 156 extends to an opening at the first end 192 of the valve stem 124. The radial port 162 is adapted to allow pressurized formulation disposed within the metering chamber 169 to pass through the radial port and into the blind hole 156 where it can be directed out of the valve 126 and into a jet orifice (i.e., jet orifice 30 of FIG. 2) of the inhaler when at least a portion of the port is disposed within the metering chamber as is further described herein.

The metering valve stem 124 can take any suitable shape or shapes and have any suitable dimensions. In one or more embodiments, the metering valve stem 124 has a constant cross-sectional area along a stem axis that extends between the first end 192 and the second end 194 of the valve stem.

The metering valve 126 also includes a reservoir seal 198 disposed adjacent to the second end 155 of the valve housing 164. As used herein, the phrase “adjacent to the second end” means that the reservoir seal 198 is disposed closer to the second end 155 of the valve housing 164 than to the first end 153 of such housing. The reservoir seal 198 can be disposed in any suitable location relative to the second end 155 of the valve housing 164. In the embodiment illustrated in FIG. 9, the reservoir seal 198 is disposed within the valve housing 164. In one or more embodiments, the reservoir seal 198 can be disposed outside of the housing 164 as is further described herein. The reservoir seal 198 is adapted to form a dynamic seal between the metering valve stem 124 and the reservoir. Further, the reservoir seal 198 can include any suitable seal or seals, e.g., the same seal described herein regarding metering valve seal member 59 of FIGS. 1-8.

The reservoir seal 198 can be retained within the housing 164 using any suitable technique or techniques. For example, the reservoir seal 198 can be friction-fit within the housing, adhered to the walls 190 of the housing, mechanically fastened to the housing, etc. In the embodiment illustrated in FIG. 9, the valve housing 164 includes a crimped portion 170 that is adapted to retain the reservoir seal 198 between the crimped portion and the second end 155 of the valve housing. The crimped portion 170 can take any suitable shape or shapes and have any suitable dimensions.

In use, the valve 126 has a primed position (FIG. 9) and an actuated position. When in the primed position, formulation from the reservoir can freely flow between the reservoir and the metering chamber 169. Further, the metering valve stem seal member 159 seals the metering chamber 169 from the outside atmosphere. An abutment 158 of the metering valve stem 124 engages with the metering valve stem seal member 159 to form a seal between the metering valve stem and the valve housing 164 when the valve is in the primed position. When in the actuated position, the metering valve stem 124 is actuated toward the second end 155 of the valve housing. In such position, an opening 181 disposed in the second end 155 of the valve housing 164 is sealed from the reservoir, thereby defining a metered volume of formulation within the metering chamber 169. Further, the metering valve stem 124 includes a flow path through the radial port 162 and the blind hole 156, thereby allowing the formulation to freely flow between the metering chamber 169 and the outside atmosphere. Such a path is formed when at least a portion of the port 162 is disposed within the metering chamber 169 when the metering valve stem 124 is actuated toward the second end 155 of the housing 164.

In one or more embodiments, the valve 126 can be urged from the actuated position to the primed position by the pressurized propellant alone and without the aid of a return spring. In contrast, other pMDI use a spring in the valve to bias the valve stem towards the primed position. Other inhalers are also known where springs are employed to assist with the sealing elements of the valve. In one or more embodiments, the sealing elements of the valve 126 are not assisted by springs. Thus, the valve 126 can act as a springless valve (i.e., neither stem bias nor seal assist springs used).

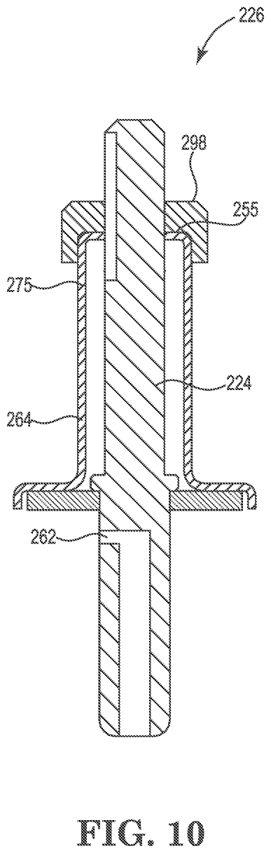

As mentioned herein, the reservoir seal 198 can be disposed in any suitable location relative to the second end 155 of the valve housing 164. For example, FIG. 10 is a cross-sectional view of another embodiment of a metering valve 226. All of the design considerations and possibilities described herein regarding the metering valve 26 of FIGS. 1-8 and the metering valve 126 of FIG. 9 apply equally to the metering valve 226 of FIG. 10. One difference between metering valve 226 of FIG. 10 and metering valve 126 of FIG. 9 is that a reservoir seal 298 is disposed on an outer surface 275 of a valve housing 264 adjacent to a second end 255 of the valve housing. The reservoir seal 298 is adapted to form a dynamic seal between metering valve stem 224 and a reservoir (e.g., reservoir 37 of FIG. 2) of an inhaler (e.g., inhaler 10 of FIG. 2) connected to the valve. The reservoir seal 298 can include any suitable seal or seals described herein, e.g., reservoir seal 198 of FIG. 9. Further, the reservoir seal 298 can be disposed on and in contact with the outer surface 275 of the valve housing 264. In one or more embodiments, one or more layers or elements can be disposed between the reservoir seal 298 and the outer surface 275 of the valve housing 264.

The reservoir seal 298 can be retained on the outer surface 275 of the valve housing 264 using any suitable technique or techniques, e.g., the seal can be friction-fit to the second end 255 of the valve housing. In one or more embodiments, the reservoir seal 298 can be attached to the second end 255 of the valve housing 264 using any suitable attachment technique, e.g., adhesives, mechanical fasteners, etc. In one or more embodiments, the reservoir seal 298 is retained on the outer surface 275 of the valve housing 264 by a valve clamp (e.g., valve clamp 74 of valve 26 of FIG. 5). The valve clamp can be disposed over the reservoir seal 298 and at least a portion of the second end 255 of the valve housing 264.

### EXAMPLES

Canister Filling/Inhaler Preparation—Examples 1 to 5

Formulations were prepared using a refillable two-part 12 g canister (Modern Combat Sports UK) equipped with a filling valve and an outlet valve. The canister was opened and a quantity of liquid containing the non-propellant components of the composition was dispensed into the open canister. The canister halves were attached to each other and the canister was filled through the filling valve with a quantity of carbon dioxide from a cylinder containing carbon dioxide using a 12 g carbon dioxide cylinder charger (Modern Combat Sports, UK). The canister was shaken and then allowed to rest for approximately 15 minutes.

The outlet valve of the filled canister was attached to a sealed manifold equipped with a metering valve as shown in FIGS. 2-7. The outlet valve was then fixed in the open position so that the canister and internal manifold volume formed a single pressurized reservoir volume. The metering valve was a type 20 DR 376/65/0 metering valve (Coster, Italy) modified by removing the mounting cup, internal gasket, external gasket and spring. A PTFE O-ring (ID 2.57 mm x CS 1.78 mm, Polymax, UK) was used to provide the external seal around the metering valve stem. The canister-manifold-metering valve assembly was fitted with an actuator.

Through Life Shot Weight—Examples 1 to 5

A filled canister was prepared as described above and connected to an inhaler as described above. Units were tested with an aluminum actuator with a plastic insert having a 0.319 mm spray orifice. The inhaler was actuated with the plume directed towards waste collection. The weight of the inhaler was determined before and after each shot and the shot weight was determined from the difference. Results are reported as the number of shots for the shot weight to reach approximate steady state, the number of shots produced at steady state, and the average and standard deviation of the shot weights at steady state.

### Example 1

Approximately 8.3 g of a 10% (w/w) ethanol in carbon dioxide composition was prepared in a 12 g canister according to the canister filling procedure. The canister was allowed to rest for approximately 15 minutes. The canister was then connected to an inhaler device as described above. The through life shot weights were determined as described above. After eleven shots the inhaler produced 78 shots with an average of 58.3+/−2.5 mg.

### Example 2

Approximately 8.2 g of a 20% (w/w) ethanol in carbon dioxide composition was prepared in a 12 g canister according to the canister filling procedure. The canister was allowed to rest for approximately 15 minutes. The canister was then connected to an inhaler device as described above. The through life shot weights were determined as described above. After three shots the inhaler produced 85 shots with an average of 62.0+/−0.5 mg.

### Example 3

A solution of beclomethasone dipropionate (Teva) in ethanol (99.5% Acros) was prepared by adding 0.23 g beclomethasone to 10 ml ethanol. A 0.87 g aliquot of the beclomethasone dipropionate in ethanol solution and 6.09 g of carbon dioxide were added to a refillable 12 g canister. The canister was shaken and then allowed to rest for approximately 15 minutes. The canister was connected to an inhaler device. The through life shot weights were determined. After three shots the inhaler produced 64 shots with an average of 64.5+/−0.5 mg.

### Example 4

A mixture of micronized fluticasone propionate (Hovione) and ethanol (100% BP/EP Hayman, Essex, UK) was prepared by adding 109 mg of fluticasone propionate to 3.95 g of ethanol. The mixture was stirred to form a homogeneous suspension. A 0.84 g aliquot of the fluticasone propionate and 6.4 g of carbon dioxide were added to a refillable 12 g canister. The canister was shaken and then allowed to rest for approximately 15 minutes. The canister was connected to the inhaler device. The through life shot weights were determined. After two shots the inhaler produced 62 shots with an average of 63.0+/−8.2 mg.

### Example 5

A mixture of micronized salbutamol sulphate (Teva API, Israel) and ethanol (100% BP/EP Hayman, Essex, UK) was prepared by adding 0.133 g of salbutamol sulphate and mixing with 7.89 g of ethanol. The mixture was stirred to form a homogeneous suspension. A 12 g canister was filled with a 0.846 g aliquot of the salbutamol sulphate (SS) in ethanol suspension, 0.862 g ethanol, and 7.142 g of carbon dioxide. The canister was shaken and then allowed to rest for approximately 15 minutes. The canister was connected to an inhaler device. The through life shot weights were determined. After three shots the inhaler produced 70 shots with an average of 64.3+/−2.7 mg.

Canister Filling/Inhaler Preparation—Example 6

Units were prepared using a refillable three-part canister (Kindeva Drug Delivery L.P., St. Paul, Minn.) equipped with a filling valve and a metering valve. The metering valve was a modified Kindeva™ Spraymiser™ valve core assembly consisting of a valve stem, spring, outer and inner seal, fitted into the canister ferrule with a metering tank and held in place with a valve retainer. A diaphragm (ID 0.094″ OD 0.347″) was punched from a 1 mm thick sheet (Hytrel 4556, Du Pont) and used to provide the external seal around the metering valve stem and a seal (ID 0.069″ OD 0.152″) was punched from the same sheet and used to provide an internal seal. Carbon dioxide was dispensed into the canister by attaching the canister halves to each other and filling through the filling valve with a quantity of carbon dioxide from a cylinder containing carbon dioxide using a 12 g carbon dioxide cylinder charger (Modern Combat Sports, UK). The canister was then fired to function using a firing block (4 shots) manufactured by Kindeva Drug Delivery L.P.

Through Life Shot Weight—Example 6

A filled canister was prepared as above. Shot weight was determined using a firing block manufactured by Kindeva Drug Delivery L.P. The inhaler was actuated with the plume directed towards waste collection. The weight of the inhaler was determined before and after each shot, and the shot weight was determined from the difference. The result is reported as the number of shots for the shot weight to reach approximate steady state, the number of shots produced at steady state, and the average and standard deviation of the shot weights at steady state.

### Example 6

A unit was prepared as described above was filled with carbon dioxide. Shot weight testing was completed on the seventh day after filling. After 1 shot, the inhaler produced 73 shots with an average 38.9+/−6.5 mg.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure.

All references and publications cited herein are expressly incorporated herein by reference in their entirety into this disclosure.

Various features and aspects of the present disclosure are set forth in the following claims.

1. A metered dose inhaler, comprising:
a reservoir containing a pressurized formulation of medicament and carbon dioxide, and equipped with a metering valve comprising a metering chamber, a metering valve stem, and a metering valve stem seal having an opening through which the metering valve stem passes to form a dynamic seal between the metering valve stem and outside atmosphere, and
wherein the metering valve stem seal has a Shore D hardness of 45 to 80 and its opening is adapted to be stretched wider by the metering valve stem passing through it than it would be absent the valve stem.

2-4. (canceled)

5. The inhaler of claim 1, wherein the metering valve stem seal does not comprise a spring.

6. The inhaler of claim 1, wherein the valve does not comprise a spring.

7-12. (canceled)

13. The inhaler of claim 1, wherein the metering valve stem seal is selected from polytetrafluoroethylene, ethylene tetrafluoroethylene, fluorinated ethylene propylene, polyethylene, polyamide, and polypropylene.

14-38. (canceled)

39. The inhaler of claim 1, wherein the metering valve further comprises:
a valve housing comprising a first end, a second end, and walls defining the metering chamber; and
a reservoir seal disposed adjacent to the second end of the valve housing, wherein the reservoir seal is adapted to form a dynamic seal between the metering valve stem and the reservoir.

40. The inhaler of claim 39, wherein the reservoir seal is disposed within the valve housing.

41. The inhaler of claim 40, wherein the valve housing further comprises a crimped portion that is adapted to retain the reservoir seal between the crimped portion and the second end of the valve housing.

42. The inhaler of claim 39, wherein the reservoir seal is disposed on an outer surface of the valve housing.

43. The inhaler of claim 42, wherein the reservoir seal is friction fit to the outer surface of the valve housing.

44. The inhaler of claim 42, wherein the reservoir seal is retained on the outer surface of the valve housing by a valve clamp that is disposed over the reservoir seal and at least a portion of the second end of the valve housing.

45. The inhaler of claim 39, wherein the valve does not include a spring.

46. A metered dose inhaler having a reservoir containing a pressurized formulation of medicament and carbon dioxide, wherein the metered dose inhaler is equipped with a valve comprising:
a valve housing having a first end, a second end exposed to pressurized formulation in the reservoir, and walls defining a metering chamber for receiving the formulation from the reservoir,
a metering valve stem located within the metering chamber, the metering valve stem having a first end exposed to atmosphere and a second end exposed to pressurized formulation in the reservoir, at least a portion of the metering valve stem proximate the first end having a diameter larger than the diameter of at least a portion of the metering valve stem proximate the second end, and
a metering valve stem seal member in contact with the first end of the valve housing and the metering valve stem, wherein the metering valve stem seal member has a Shore D hardness of 45 to 80,
the valve having a primed position wherein the formulation can freely flow between the reservoir and the metering chamber, and wherein the metering valve stem seal member seals the metering chamber from the outside atmosphere, and
the valve having an actuated position wherein the second end of the metering valve stem seals against the second end of the valve housing to seal the metering chamber from the reservoir, thereby defining a metered volume of formulation within the metering chamber, and wherein the metering valve stem comprises a flow path allowing the formulation to freely flow between the metering chamber and the outside atmosphere.

47. The inhaler of claim 46, wherein the valve is urged from the actuated position to the primed position solely by a force of the pressurized formulation.

48. The inhaler of claim 46, wherein the metering valve stem seal does not comprise a spring.

49. The inhaler of claim 46, wherein the valve does not comprise a spring.

50. A metered dose inhaler having a reservoir containing a pressurized formulation of medicament and carbon dioxide, wherein the inhaler is equipped with a valve comprising:
a valve housing having a first end, a second end with an opening exposed to pressurized formulation in the reservoir, and walls defining a metering chamber for receiving the formulation from the reservoir,
a metering valve stem located within the metering chamber, the metering valve stem having a first end exposed to atmosphere and a second end exposed to pressurized formulation in the reservoir, at least a portion of the metering valve stem proximate the first end having a diameter larger than the diameter of at least a portion of the metering valve stem proximate the second end, and
a metering valve stem seal member in contact with the first end of the valve housing and the metering valve stem, wherein the metering valve stem seal member has a Shore D hardness of 45 to 80 and does not comprise a spring,
the valve having a primed position wherein the formulation can freely flow between the reservoir and the metering chamber, and wherein the metering valve stem seal member seals the metering chamber from the outside atmosphere, and
the valve having an actuated position wherein the opening at the second end of the valve housing is sealed from the reservoir, thereby defining a metered volume of formulation within the metering chamber, and wherein the metering valve stem comprises a flow path allowing the formulation to freely flow between the metering chamber and the outside atmosphere.

51. The inhaler of claim 50, wherein the second end of the metering valve stem seals against the second end of the valve housing to seal the metering chamber from the reservoir when the valve is in the actuated position.

52. The inhaler of claim 50, wherein the valve is urged from the actuated position to the primed position solely by a force of the pressurized formulation.

53. The inhaler of claim 50, wherein the valve does not comprise a spring.

54. The inhaler of claim 50, wherein the metering valve stem seal is selected from polytetrafluoroethylene, ethylene tetrafluoroethylene, fluorinated ethylene propylene, polyethylene, polyamide, and polypropylene.
